(19) United States
(12) Patent Application Publication
(10) Pub. No.: US 2017/0128743 A1
(43) Pub. Date: May 11, 2017

(54) SMART BED FOR RADIATION THERAPY

(71) Applicant: KOREA ATOMIC ENERGY RESEARCH INSTITUTE, Daejeon (KR)

(72) Inventors: Seungho KIM, Daejeon (KR); Kyungmin JEONG, Daejeon (KR); Yeonggeol BAE, Daejeon (KR); Hyeonseok NA, Chungcheongbuk-Do (KR)

(73) Assignee: KOREA ATOMIC ENERGY RESEARCH INSTITUTE, Daejeon (KR)

(21) Appl. No.: 15/249,891

(22) Filed: Aug. 29, 2016

(30) Foreign Application Priority Data
Nov. 10, 2015 (KR) 10-2015-0157685

(51) Int. Cl. A61N 5/10; A61G 13/06; A61B 6/04

(52) U.S. Cl. CPC A61N 5/10; A61B 6/0407; A61B 6/0457; A61B 6/0464; A61G 13/06; A61N 2005/1097

(57) ABSTRACT

A smart bed for a radiation therapy includes a rest to which radiation is applied, a support structure having one end fixed to the ground and having four columns disposed to be spaced apart from one another, centered around the rest, four connection parts extending from one side of each of the columns and connected to each vertex of the rest, and lengthened or shortened through a linear actuator provided on one side thereof, and a controller independently controlling each of the linear actuators to implement movement of the rest, wherein rotational and translational movement of the rest is implemented such that radiation is applied to each position of an upper surface and a lower surface of the rest.

### CROSS-REFERENCE TO RELATED APPLICATION

Pursuant to 35 U.S.C. §119(a), this application claims the benefit of earlier filing date and right of priority to Korean Application No. 10-2015-0157685, filed on Nov. 10, 2015, the content of which is incorporated by reference herein in its entirety.

### FIELD OF THE INVENTION

The present disclosure relates to a smart bed for radiation therapy, which applies radiation to a target.

### BACKGROUND OF THE INVENTION

In order to apply radiation to a target for radiation therapy, a bed for stably positioning the target thereon is required. A bed may fix the target not to be moved, and in general, a bed aimed for radiation therapy is manufactured to have a joint-type structure in the form of a cantilever. However, the joint-type structure in the form of a cantilever is heavy in weight and since it should be installed on the floor, it is impossible to apply radiation from a lower side of a bed.

Recently used robot vertical multi-joint type radiation therapy equipment is difficult to prevent occurrence of a position in which it is impossible to apply radiation in a region in which a bed is driven, moved, and operated to multiple degrees.

### SUMMARY OF THE INVENTION

Therefore, an aspect of the detailed description is to provide a novel structure of a smart bed for a radiation therapy in which various movements may be implemented.

Another aspect of the detailed description is to provide a structure of a smart bed for a radiation therapy, capable of applying radiation to any position including a lower side of a smart bed for a radiation therapy.

To achieve these and other advantages and in accordance with the purpose of this specification, as embodied and broadly described herein, a smart bed for a radiation therapy may include: a rest to which radiation is applied; a support structure having one end fixed to the ground and having four columns disposed to be spaced apart from one another, centered around the rest; four connection parts extending from one side of each of the columns and connected to each vertex of the rest, and lengthened or shortened through a linear actuator provided on one side thereof; and a controller independently controlling each of the linear actuators to implement movement of the rest, wherein rotational and translational movement of the rest is implemented such that radiation is applied to each position of an upper surface and a lower surface of the rest.

According to an example related to the present disclosure, the controller may select two certain linear actuators and transmit an operational signal such that the rest is rotated on the basis of an X axis or a Y axis.

According to an example related to the present disclosure, the controller may select all the linear actuators and transmit an operation signal, for a vertical translational movement of the rest.

According to an example related to the present disclosure, each of the connection parts may have an actuator fixing fastening member formed to surround at one end so as to be fixed to the column.

According to an example related to the present disclosure, each of the connection parts may have a joint on one side for a rotational movement on the basis of a Z axis of the rest.

According to an example related to the present disclosure, each of the connection parts may be configured as a rest connection member supporting the rest and transmitting tensile force to allow the rest to be positioned on the ground.

According to an example related to the present disclosure, the smart bed may further include: a radiation applying unit positioned at an upper end and a lower end of the rest and applying radiation.

According to an example related to the present disclosure, the smart bed may further include: a rest support unit having one end attached to the ground and the other end supporting a lower portion of the rest in order to reduce swinging of the rest.

According to an example related to the present disclosure, the smart bed may further include: a calculation processing unit inputting a set value for controlling the rest to the controller and having a display displaying the input value and a current state of the rest.

Further scope of applicability of the present application will become more apparent from the detailed description given hereinafter. However, it should be understood that the detailed description and specific examples, while indicating preferred embodiments of the invention, are given by way of illustration only, since various changes and modifications within the scope of the invention will become apparent to those skilled in the art from the detailed description.

### BRIEF DESCRIPTION OF THE DRAWINGS

The accompanying drawings, which are included to provide a further understanding of the invention and are incorporated in and constitute a part of this specification, illustrate exemplary embodiments and together with the description serve to explain the principles of the invention.

In the drawings:

FIG. 1 is a perspective view illustrating a smart bed for a radiation therapy.

FIG. 2 is a perspective view illustrating a smart bed for a radiation therapy including a radiation apply unit.

FIG. 3 is a side view of the smart bed for a radiation therapy of FIG. 1 or 2.

FIG. 4 is an enlarged view of a portion “A” of FIG. 3.

FIG. 5 is an enlarged view of a portion “B” of FIG. 3.

FIG. 6 is a perspective view illustrating another embodiment of a smart bed for a radiation therapy of FIGS. 1 to 5.

### DETAILED DESCRIPTION OF THE INVENTION

Description will now be given in detail of the exemplary embodiments, with reference to the accompanying drawings. For the sake of brief description with reference to the drawings, the same or equivalent components will be provided with the same reference numbers, and description thereof will not be repeated.

Hereinafter, a bed for a radiation therapy related to the present disclosure will be described in detail.

Hereinafter, the present disclosure will be described in detail with reference to the accompanying drawings, in which like numbers refer to like elements throughout although the embodiments are different. As used herein, the singular forms “a”, “an” and “the” are intended to include the plural forms as well, unless the context clearly indicates otherwise.

A smart bed 100 or 200 for a radiation therapy refers to an apparatus aiming at applying radiation to a target, which is able to apply radiation to a target positioned on a rest (or a bed) and the rest may refer to a medical bed (couch) for treating a patient. Here, the target refers to an object or a person positioned on a radiation bed. The present disclosure relates to a bed using radiation, and a target may be considered as a person requiring a radiation therapy. That is, in the present disclosure, a target may refer to a tumor patent to be treated. However, the present disclosure is not limited thereto.

In order to apply radiation to each position of a target, radiation needs to be applied even to a lower side of the smart bed 100 for a radiation therapy, as well as to an upper side of the smart bed 100 for a radiation therapy.

The smart bed 100 for a radiation therapy includes a rest 110, a support structure 120, a connection part 130, a controller (not shown), a radiation applying unit 150, a rest support unit 160, and a calculation processing unit (not shown).

The present disclosure provides a structure in which the rest 110 is supported by the support structure 120 such that radiation emitted from the radiation applying unit 150 is applied to a lower portion of the smart bed 100.

The rest 110, referring to a device allowing a person which is to be subjected to a radiation therapy to lie down thereon, may perform various functions for a radiation therapy, unlike general beds. A radiation may transmit through the rest 110 so as to be applied to a diseased area of the person lying on the rest 110, and generally has a rectangular shape. The rest 110 is formed of a synthetic resin material allowing a radiation to be transmitted therethrough, and may have a weight reduced using a porous material and a composite material. The rest 110 used in the present disclosure is merely illustrative and may have various other shapes than that illustrated in the drawing.

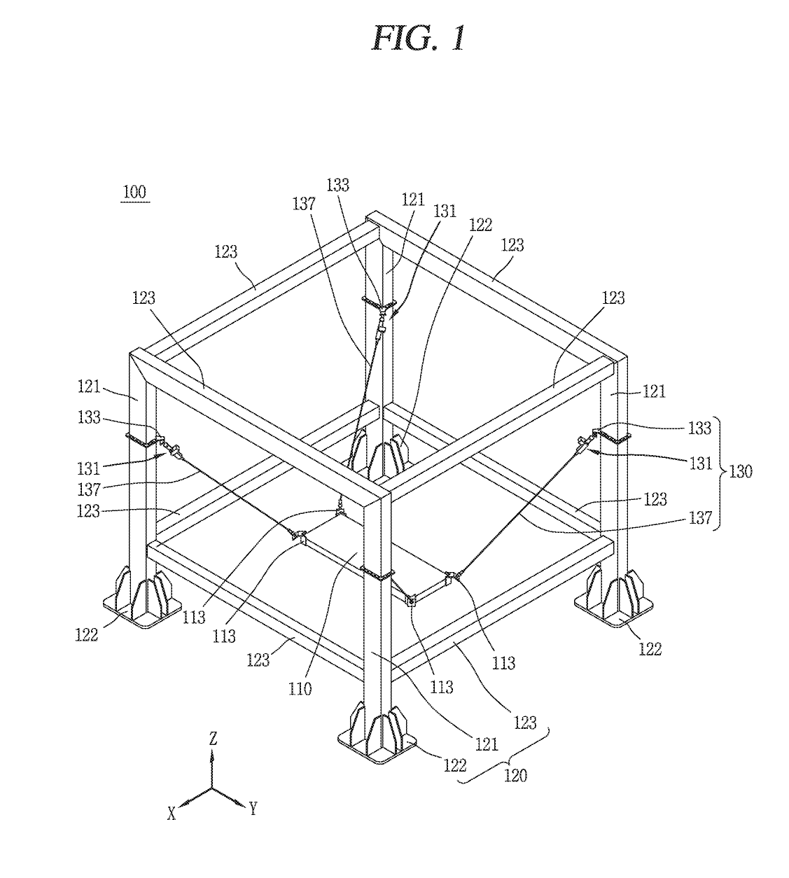

Referring to FIG. 1, the support structure 120 supports the rest 110, forms an overall shape of the smart bed 100 for a radiation therapy, and includes a pillar 121, a ground fixing part 122 for fixing the pillar 121 on the ground, and a crossbeam 123 connecting the pillars 121.

The pillar 121, the ground fixing part 122, and the crossbeam 123 are generally formed of a hard metal material but not limited thereto.

The pillar 121, serving to support the rest 110, is fixed on the ground. The pillar 121 is screw-coupled to the ground through the ground fixing part 122 such that the pillar 121 is fixed upwardly. As illustrated in FIG. 1, the pillar 121 has a quadrangular shape. The ground fixing part 122 has a structure supporting each corner of the pillar 121 having a shape of a vertical bar, but the ground fixing part 122 may have various other shapes as long as it enables the pillar 122 to be positioned in a state of being fixed on the ground. The pillars 121 are disposed to be spaced apart from each other by a predetermined interval, centered on the rest 110. In the present disclosure, the support structure 120 includes four pillars 121 facing each other.

The crossbeams 123 serve to stably distribute the weight generated as the columns 124 support the rest 110. As illustrated in FIG. 1, the crossbeams 123 serve to link the pillars 121 adjacent to each other in upper and lower portions of the pillars 121. However, a position and a shape of the crossbeams 123 are not limited to those illustrated in FIG. 1.

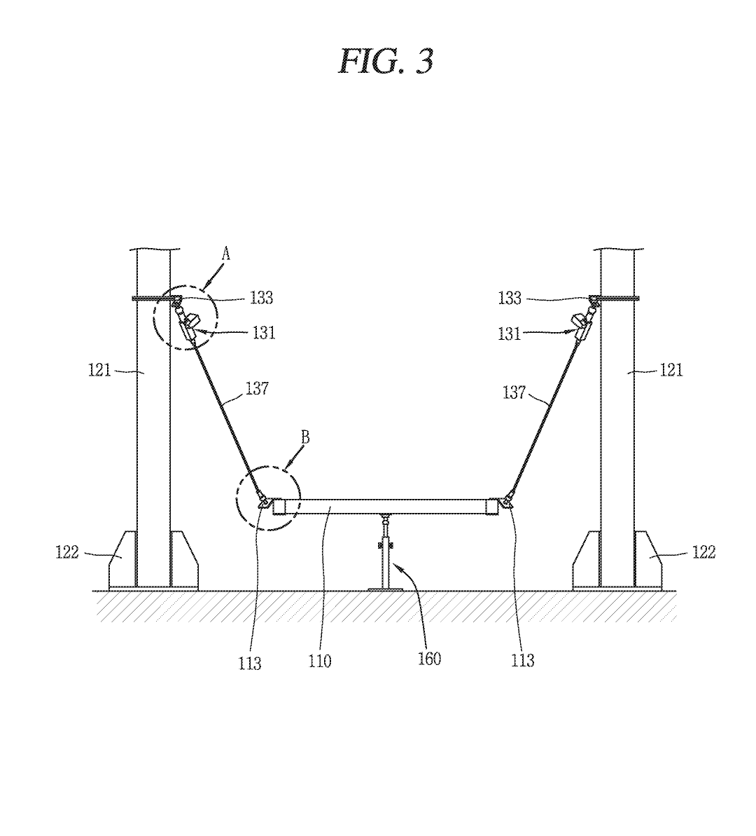

The connection part 130 extends from one side of each pillar 121 and is connected to each vertex of the rest 110 to support the rest 110, and includes a linear actuator 131 provided on one side in order to extend or shorten in a length direction. Supporting of the rest 110 by the connection part 130 may be checked in FIG. 5 as an enlarged view of a portion “B” of FIG. 3.

Referring to FIG. 5, a rest fastening member 113 is positioned in each vertex portion of the rest 110 such that it is supported by the connection part 130. The rest fastening member 113 may be formed of a metal, configured to cover the vertex portion of the rest 110, and have a hole positioned in a portion protruding from an upper end thereof to allow the connection part 130 to be coupled therethrough. The connection part 130 supports each vertex portion of the rest 110 in such a manner that one side thereof is inserted into the hold positioned in the protrusion portion of the rest fastening member 113.

A total of four connection parts 130 may be provided to connect the rest 110 to the support structure 120. The connection part 130 is a mechanical structure that may be able to expand or contract, serving to transfer force to the rest 110. The connection part 130 may include a rest connection member 137, a linear actuator 131, and an actuator fixing fastening member 133.

The rest connection member 137 serves to connect the rest fastening member 113 positioned in each vertex portion of the rest 110 and the actuator fixing fastening member 133 positioned in the pillar 121. The rest connection member 137 is formed of steel and serves to transmit force to the bed according to an operation of the linear actuator 131. The rest connection member 137 may be formed of a steel wire, a steel plate, or a bar having a large slenderness ratio.

Here, the steel wire refers to a wire rod formed of steel. In the present disclosure, the rest connection member 137 may be mild drawn wire, a hard steel wire, or a piano wire. The mild drawn wire, generally called an iron wire, has a carbon content ranging from about 0.06 to 0.25% and tensile strength ranging from about 35 to 70 kg/mm². The hard steel wire is manufactured by cooling a wire rod obtained by rolling acid open hearth steel or electric furnace steel to have a diameter ranging from 5 to 5 mm. Tensile strength of the hard steel wire varies depending on components, but in case of the same type of materials, tensile strength is greater as a diameter is smaller. The piano wire refers to carbon steel wire heat-treated while being drawn to have a sorbite tissue.

Steel plate refers to a plate formed of steel, and is divided into a thick plate, a medium plate, and a thin plate according to plate thicknesses. Specifically, a plate having a thickness of 6 mm or greater is classified as a thick plate, a plate having a thickness ranging from 1 mm to 6 mm is classified as a medium plate, and a plate having a thickness of 1 or less is classified as a thin plate. Steel plate may be classified as hot rolled steel used in architecture, bridge, vehicles, and ships, rolled steel for a welding structural purpose, having excellent weldability, non-tempered high tensile steel having high yield point, and quenched and tempered high strength steel. In the present disclosure, the rest connection member 137 may be manufactured using each steel plate.

The slenderness ratio refers to a ratio between a radius (k) of gyration of area of a pillar and a length (l) of the pillar, which is used to calculate buckling strength. In the present disclosure, a rest connection member having a low slenderness ratio, and here, when a slenderness ratio is low, it means that buckling occurs readily.

An actuator refers to a device for driving a movement of a mechanical device driving unit. The actuator may be, for example, a servo motor, a step motor, a hydraulic motor, a hydraulic cylinder, or a pneumatic cylinder. The actuator may include a gear, a ball thread, and a link as components, and a gap and mechanical strength between fastening elements affect accuracy and response performance of a system. In the present disclosure, a linear actuator, a sort of an actuator, is used. The linear actuator is able to pull or push the rest connection member 137, serving to transmit force to the rest 110 connected to one end of the rest connection member 137.

The linear actuator 131 is disposed on one side of the connection part 130. In detail, the linear actuator 131 is connected to one side of the actuator fixing fastening member 133 surrounding the column 121.

The linear actuator 131 may receive power through a power supply unit, and the linear actuators 131 respectively connected to the connection parts 130 may be independently controlled by a controller (not shown). When independent driving of the linear actuator 131 is used, various movements of the rest 110 connected to one side of the connection part 130 may be implemented.

The linear actuator 131 used in the present disclosure may not be limited in type or specific shape as long as it can transmit tensile force or compressive force to the rest 110 in a length direction through the rest connection member 137. The linear actuator 131 may include a linear motor. The linear actuator 131 illustrated in FIG. 1 is an example, and the linear actuator 131 may have various other shapes.

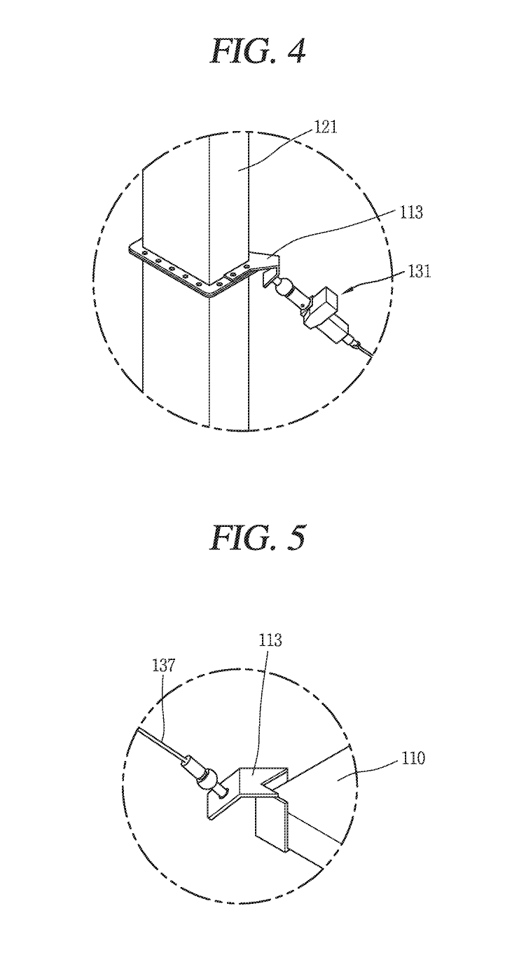

FIG. 3 is a side view of the smart bed 100 for a radiation therapy and FIG. 4 is an enlarged view of a portion “A” of FIG. 3.

As illustrated in FIG. 4, the actuator fixing fastening member 133 has a shape surrounding the column 121 and tightly attached to an outer circumference of the column 121 so as to be fixed to the column 121. The actuator fixing fastening member 133 may have two components, ad the two components may be firmly fixed to each other through a plurality of recesses that can be screw-coupled. The actuator fixing fastening member 133 may be fixed to the column 121 to correspond to a change in a shape of the column 121. One side of the actuator fixing fastening member 133 may be connected to the linear actuator 131. The actuator fixing fastening member 133 is generally formed of a metal, but a material of the actuator fixing fastening member 133 is not limited thereto and the actuator fixing fastening member 133 may be fixed to the column 121 through a screw fastening recess.

The controller (not shown) serves to control an operation of the linear actuator 131. The controller (not shown) controls the linear actuator 131 positioned on one side of each of the connection parts 130 supporting the rest 110, thus implementing a movement of the rest 110. When an equipment operator inputs a set value for implementing a movement of the rest 110 in order to apply radiation to each position of the rest 110, the controller (not shown) transmits a signal for operating each of the linear actuators 131.

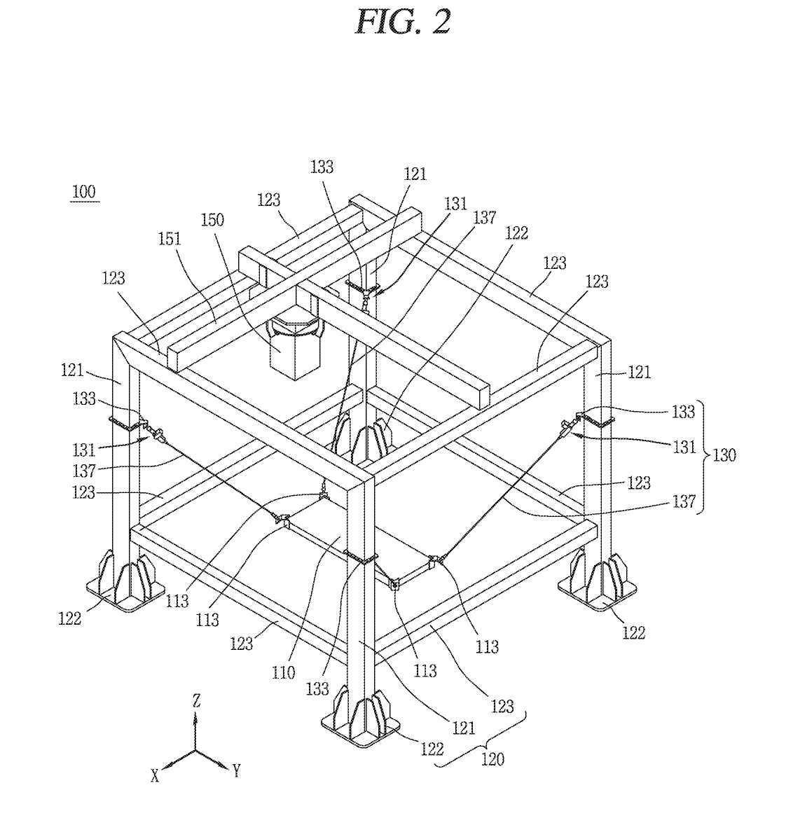

FIG. 2 illustrates the smart bed 100 for a radiation therapy in which the radiation applying unit 150 is positioned in an upper end of the support structure 120.

In FIG. 2, the radiation applying unit 150, which serves to apply radiation to the rest 110, may be positioned in an upper end or a lower end of the support structure 120. The radiation applying unit 150 may be configured as a robot applying radiation. The radiation applying unit 150 may be fixed to positions of the crossbeams 123 of the support structure 120 in order to stably apply radiation toward a target positioned on the rest 110. For example, as illustrated in FIG. 2, the radiation applying unit 150 may be fixed through screw fastening, or the like, to positions of the crossbeams 123 of the support structure 120 through a radiation applying frame 151.

FIG. 3 is a side view of the smart bed 100 for a radiation therapy. The smart bed 100 for a radiation therapy may further include the rest support unit 160.

One end of the rest support unit 160 is fixedly attached to the ground and the other end thereof supports a lower portion of the rest 110. Accordingly, the rest support unit 160 may reduce swinging of the rest 110. Supporting a central lower portion of the rest, the rest support unit 160 may more stably implement a movement of the rest 110 by the linear actuator 131. This is directly connected to safety of a person positioned on the rest 110.

The rest support unit 160 may have various shapes and may have a height adjusting unit for adjusting a height in order to implement a stable operation of the rest 110 even when the rest 110 makes a vertical translational movement.

The calculation processing unit (not shown) may input a set value for controlling the rest 110 to the controller (not shown) and may have a display for displaying the input value and a current position state of the rest 110.

Hereinafter, a method for implementing various movements of the rest 110 will be described. A target positioned on the rest 110 requires application of radiation, and in order to apply radiation to each position of the rest 110, preferably, radiation is applied also to a lower portion of the rest 110, as well as to an upper portion of the rest 110. In the present disclosure, since radiation is applied to each position below the rest 110, a range of an irradiation angle for a radiation therapy may be expanded and efficiency of a radiation therapy and effectiveness of a treatment may be increased. Also, an effect of reducing a spatial restriction regarding an installation position of a radiation therapy robot or the radiation applying unit 150 may be reduced.

Movement of the rest 110 that may be implemented in the present disclosure will be described with reference to FIGS. 1 to 3. The linear actuator 131 is positioned on one side of each of the connection part 130, and as power is supplied, the linear actuator 131 is operated and tensile force or compressive force is applied to the connection part 130.

Compressive force or tensile force applied to the connection part 130 is transmitted to the rest 110 through the rest fastening member 113 connected to one end of the connection part 130. Movement of the rest 110 is implemented by the compressive force or tensile force.

Also, each of the linear actuators 131 positioned on one side of each of the four connection parts 130 may be independently operated by the controller (not shown), and various movements of the rest 110 may be implemented through driving of the independent linear actuator 131.

X, Y, and Z axes displayed in FIGS. 1 and 2 refer to rotation reference axes of the rest 110. The smart bed 100 for a radiation therapy may be rotated on the basis of the X axis, Y axis, and Z axis.

A rotational movement of the rest 110 on the basis of the X axis or the Y axis may be made by operating certain two linear actuators 131. Through an operation of the certain two linear actuators 131, the rest 110 may be rotated on the basis of the X axis or the Y axis.

For example, when compressive force is provided to the connection part 130 through operations of the two linear actuators 131 positioned on the right in relation to the rest 110, the right side of the rest 110 is lifted up by the connection part 130, rotating in the X axis. Similarly, when tensile force is provided to the connection part 130 through an operation of the two linear actuators 131 provided on the right side in relation to the rest 110, the right side of the rest 110 is moved downwardly by the connection part 130, rotating on the basis of the X axis.

The rest 110 may be stably rotated by the rest support unit 160 supporting the rest 110 upwardly. Since the rest support unit 160 supports the center of the rest 110, the rest 110 may be rotated even with the operation of the linear actuator 131, while minimizing an impact.

Also, since tensile force and compressive force or compressive force and tensile force are applied to the left and right of the rest 110 by operating the two linear actuators 131 positioned on the right side in relation to the rest 110 and the two linear actuators 131 positioned on the left side in relation to the rest 110, a rotation angle of the rest 110 may be further increased. Here, however, since a person is positioned on the rest 110, an angle at which the rest 110 is rotated is set not to exceed 5°.

Similarly, a rotational movement of the rest 110 in relation to the Y axis may be made by applying a method for rotating the rest 110 in relation to the X axis. The rest 110 may be rotated on the basis of the Y axis by tensile force and compressive force applied to the two connection parts 130 positioned on the longer side corner of the rest 110.

Rotational movement of the rest 110 in relation to the Z axis refers to rotating the rest 110 positioned at a predetermined height in relation to the ground in a clockwise direction or in a counterclockwise direction. Rotational movement of the rest 110 in the Z axis direction may be implemented through a universal joint (not shown) or a ball joint (not shown) positioned on one side of the connection part 130. Rotation of the rest 110 in the Z axis direction is also set to be made within a range of substantially 5° to ensure safety of a patient on the rest 110.

The rest 110 may make a vertical translational movement by operating all the linear actuators 131 positioned in the four connection parts 130.

When the rest 110 positioned on the ground is intended to be lifted or lowered by a predetermined height, an equipment operator may input a corresponding set height to the calculation processing unit (not shown), and all the linear actuators 131 positioned in the four connection parts 130 may be operated upon receiving a power signal by the controller (not shown) according to the set height.

When all the linear actuators 131 are operated, the connection part 130 may transmit tensile force or compressive force to vertices of the rest 110 through the rest fastening member 113. When tensile force is applied to the rest 110 through the connection part 130, the rest 110 may be lifted, and when compressive force is applied to the rest 110 through the connection part 130, the rest 110 is lowered. Lifting or lowering of the rest 110 may be performed within a range of substrate 30 centimeters.

Stability of the lowering and lifting movement of the rest 110 is guaranteed by the rest support unit 160 supporting the rest 110. As the rest 110 is lifted or lowered, a length of the rest support unit 160 is lengthened or shortened to thus support the rest 110 to be stably operated.

As described above, the smart bed 100 for a radiation therapy may perform a translational movement in the X, Y, and Z-axis directions and in a vertical direction, and a rotational angle or a vertical movement distance thereof may be adjusted.

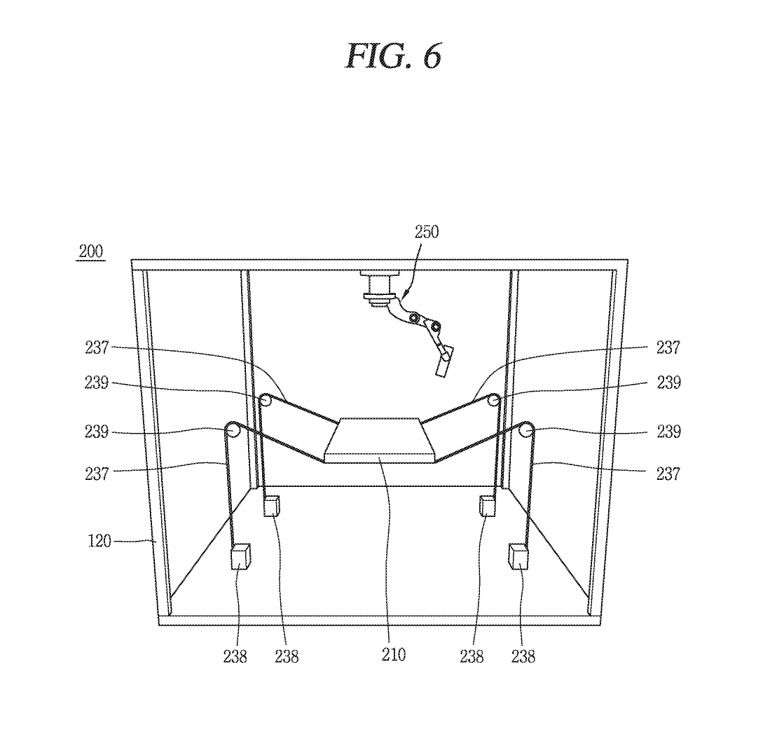

FIG. 6 is a view illustrating another embodiment of the smart bed 200 for a radiation therapy, in which a rest 210 is moved by winding or unwinding a rest connection member 237. Unlike the smart bed 100 for a radiation therapy of FIGS. 1 to 5, the smart bed 200 for a radiation therapy of FIG. 6 does not have the column 121 and the actuator fixing fastening member 133 attached to the column 121. However, the smart bed 200 for a radiation therapy of FIG. 6 has characteristics of having a rest connection member fixing unit 239 and a winding device 238.

Each of the rest connection member 237 supporting the rest 210 may implement various movements of the rest 210 through the winding device 238 fixed to the bottom surface by passing through the rest connection member 239. Since a radiation applying unit 250 is positioned above the smart bed 200 for a radiation therapy, the radiation applying unit 250 is able to apply radiation to the rest 210.

As described above, the smart bed for a radiation therapy is not limited to the configuration and method described above, but the entirety or a portion of the embodiments may be selectively combined to be configured into various modifications.

The foregoing embodiments and advantages are merely exemplary and are not to be considered as limiting the present disclosure. The present teachings can be readily applied to other types of apparatuses. This description is intended to be illustrative, and not to limit the scope of the claims. Many alternatives, modifications, and variations will be apparent to those skilled in the art. The features, structures, methods, and other characteristics of the exemplary embodiments described herein may be combined in various ways to obtain additional and/or alternative exemplary embodiments.

As the present features may be embodied in several forms without departing from the characteristics thereof, it should also be understood that the above-described embodiments are not limited by any of the details of the foregoing description, unless otherwise specified, but rather should be considered broadly within its scope as defined in the appended claims, and therefore all changes and modifications that fall within the metes and bounds of the claims, or equivalents of such metes and bounds are therefore intended to be embraced by the appended claims.

1. A smart bed for a radiation therapy, the smart bed comprising:
a rest to which radiation is applied;
a support structure having one end fixed to the ground and having four columns disposed to be spaced apart from one another, centered around the rest;
four connection parts extending from one side of each of the columns and connected to each vertex of the rest to support the rest, and lengthened or shortened through a linear actuator provided on one side thereof; and
a controller independently controlling each of the linear actuators to implement movement of the rest,
wherein rotational and translational movement of the rest is implemented such that radiation is applied to each position of an upper surface and a lower surface of the rest.

2. The smart bed of claim 1, wherein the controller selects two certain linear actuators and transmits an operational signal such that the rest is rotated on the basis of an X axis or a Y axis of the rest.

3. The smart bed of claim 1, wherein the controller selects all the linear actuators and transmits an operation signal, for a vertical translational movement of the rest.

4. The smart bed of claim 1, wherein each of the connection parts has an actuator fixing fastening member formed to surround the column at one end of the actuator fixing fastening member so as to be fixed to the column.

5. The smart bed of claim 1, wherein each of the connection parts has a joint on one side thereof for a rotational movement on the basis of a Z axis of the rest.

6. The smart bed of claim 1, wherein each of the connection parts has a rest connection member supporting the rest and transmitting tensile force to allow the rest to be positioned on the ground.

7. The smart bed of claim 1, further comprising:
a radiation applying unit positioned at an upper end and a lower end of the rest and applying radiation.

8. The smart bed of claim 1, further comprising:
a rest support unit having one end attached to the ground and the other end supporting a lower portion of the rest in order to reduce swinging of the rest.

9. The smart bed of claim 1, further comprising:
a calculation processing unit inputting a set value for controlling the rest to the controller and having a display displaying the input value and a current state of the rest.
